(19) United States
(12) Patent Application Publication
(10) Pub. No.: US 2008/0076827 A1
(43) Pub. Date: Mar. 27, 2008

(54) Treatment of Inflammatory Bowel Disease

(76) Inventors: John Brew, Essex (GB); Robin Mark Bannister, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/575,995

(22) PCT Filed: Oct. 20, 2005
(86) PCT No.: PCT/GB05/04038

§ 371 Date: Jun. 5, 2007

(30) Foreign Application Priority Data
Oct. 27, 2004 (GB) 0423927.3

(51) Int. Cl. A61K 31/19; A61P 1/00

(52) U.S. Cl. 514/568

(57) ABSTRACT

Actarit or a salt thereof is useful for the treatment of inflammatory bowel disease.

### FIELD OF THE INVENTION

This invention relates to the treatment of inflammatory bowel disease (IBD).

### BACKGROUND OF THE INVENTION

Inflammatory bowel diseases are serious and morbid conditions which can involve the whole GI tract and which include conditions such as ulcerative colitis and Crohn's disease. In particular, colitis (inflammation of the colon) exists in a number of different forms, including pseudomembranous colitis, haemorrhagic colitis, haemolytic-uraemic syndrome colitis, collagenous colitis, ischaemic colitis, radiation colitis, drug and chemical-induced colitis and diversion colitis. These conditions are associated with distressing symptoms, progressive morbidity and, if left untreated, mortality. Symptoms can include chronic diarrhea, abdominal pain/cramping, blood in stools, reduced appetite, weight loss and fever.

The aetiology of IBD is currently unknown, but is thought to have significant environmental and genetic inputs. Most patients are diagnosed between the ages of 15 to 35. Ulceration, strictures, fistulas and anal fissures are all complications that can lead to resective/reconstructive surgery to repair damage or remove diseased portions, all of which leads to further morbidity. If untreated, these complications can lead to local and then systemic infections originating from the gut flora, some of which can be fatal. IBD has a relapsing remitting profile of disease progression and is treated according to the severity of these relapses.

At present, mild IBD events are treated using drugs such as sulphasalasine or mesalamine (5-ASA). These drugs are also used chronically to control the disease between relapses. More severe relapses are treated using agents such as corticosteroids, e.g. hydrocortisone, prednisolone and budesonide. In particularly severe bouts, immunosuppressants such as azathioprine, 6-mercaptopurine, methotrexate and cyclosporin are utilised. In Crohn's disease, neutralising antibodies (infliximab) may be used. Infections associated with these diseases are often treated with antibiotics such as metronidazole, ciprofloxacin and augmentin. These therapies are non-ideal: they are either sub-maximally efficacious or efficacious and burdened with problematic side-effects, or have difficult routes of delivery.

Actarit, i.e. 4-(acetylamino)benzeneacetic acid, is a drug which is currently used in Japan for the treatment of early stage rheumatoid arthritis. Actarit has been proven to be efficacious in a number of clinical trials, reducing morning stiffness, joint pain scores and inflammatory mediators, and improving quality of life scores.

### SUMMARY OF THE INVENTION

It has now been found that actarit and its salts are useful in the treatment of inflammatory bowel disease (IBD).

### DESCRIPTION OF PREFERRED EMBODIMENTS

Salts of actarit that may be used in this invention include salts derived from inorganic or organic bases, for example alkali metal salts such as magnesium or calcium salts, and organic amine salts such as morpholine, piperidine, dimethylamine or diethylamine salts.

The drug may be delivered in various formulations, e.g. via the oral, intravenous, parenteral, rectal (enema, foam), vaginal, topical, sublingual, pulmonary or intranasal route. The formulation may be in conventional release, controlled release or timed release form, e.g. enterically coated. The oral formulation may be such to allow the release of actarit to the local area of late ileal or colonic inflammation.

The drug may be delivered in combination with other therapies that are used to alleviate the symptoms of inflammatory bowel disease. Drugs known for this purpose include steroids, salicylate drugs (e.g. 5-aminosalicylic acid, sulphasalazine), immunosuppressants (e.g. azathioprine, 6-mercaptopurine) and antibiotics (e.g. metronidazole, ciprofloxacin, augmentin). The use of actarit in combination therapy may allow reduction of toxic immunosuppressants (e.g. steroid sparing).

The amount of actarit or its salts to be used in therapy can be selected by one of ordinary skill in the art, depending on the usual factors such as the condition of the patient, the severity of the condition to be treated, the type of formulation to be administered and the route of administration. When given to humans, a suitable dosage of actarit given by the oral or rectal route is 1-5000 mg.

The following tests illustrate the invention, in conjunction with the accompanying drawings, in which:

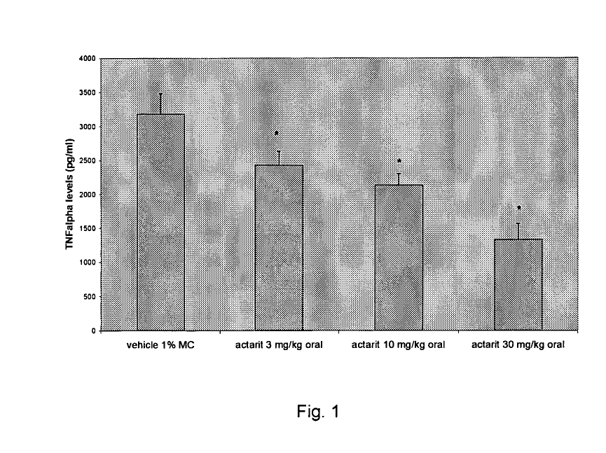

FIG. 1 shows the effect of actarit on LPS-induced TNF alpha release in mice; and

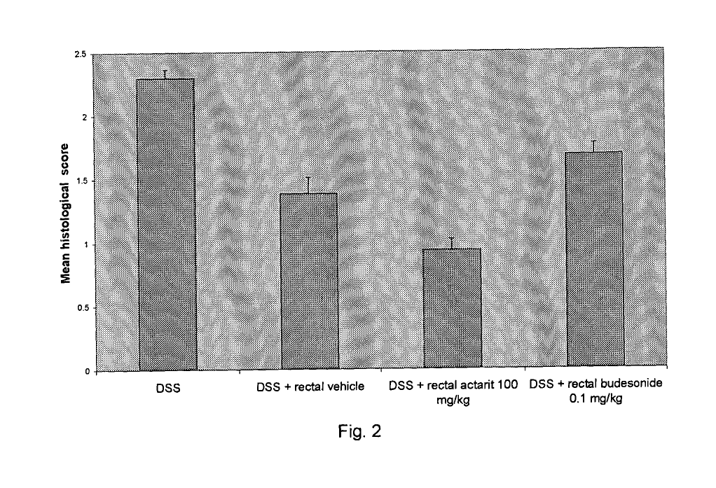

FIG. 2 shows the effect of actarit in a model of IBD.

### LPS Mouse

7 week-old Balb C ByJ mice (24-28 g) were administered, either by i.p. (5 ml/kg) or oral (10 ml/kg) administration, with vehicle or test article. 30 minutes later these animals were challenged with an intraperitoneal injection of 1 mg/kg LPS. 2 hours after LPS challenge, blood samples were collected under light isoflurane anaesthesia into normal tubes by retro-orbital puncture. Samples were allowed to clot at room temperature and then spun at 6000 g for 3 min at 4° C. Serum was stored at −20° C. until use. Serum TNFα and IL-10 levels were analysed in duplicate by ELISA technique.

Results are shown in FIG. 1. The demonstrate that, when administered via the oral route, actarit gave a dose-dependant inihibition of TNFalpha levels after intraperitoneal LPS administration.

### Dextran Sulphate-Induced IBD Model

8-10 week-old BDF1 male mice (˜30 g) were housed in normal conditions. At the start of the study, normal water was exchanged for a 2.5% dextran sulphate solution to induce colonic inflammation. Concurrently actarit and the positive control budesonide were administered rectally twice a day for 5 days. On day 6 the animals were sacrificed and the large intestine was removed. The lower two-thirds were assessed for histological severity (scoring system; 1 mild to 4 severe).

Results are shown in FIG. 2. They demonstrate that, when administered via the rectal route, actarit can ameliorate pathological and histological lesions. In particular, actarit reduced gross histological scores induced by dextran sulphate in the drinking water of BDF1 mice, over and above the effect seen for 0.1 mg/kg rectal budesonide.

In summary, actarit has been demonstrated to act as a TNF alpha inhibitor, and this has translated into efficacy in the dextran sulphate model of inflammatory bowel disease.

1. A method for the treatment of inflammatory bowel disease, wherein said method comprises administering, to a patient in need of such treatment, actarit, or a salt thereof.

2. The method, according to claim 1, wherein the disease is ulcerative colitis.

3. The method, according to claim 1, wherein the disease is Crohn's disease.

4. The method, according to claim 1, wherein the patient is also administered another agent used to treat inflammatory bowel disease.

5. The method, according to claim 4, wherein said another agent is selected from the group consisting of steroids, salicylate drugs, immunosuppressants, antibiotics and Crohn's disease-neutralising antibodies.

6. The method according to claim 5, wherein said another agent is sulphasalasine, mesalamine, hydrocortisone, prednisolone, budesonide, azathioprine, 6-mereaptopurine, methotrexate, cyclosporine or infliximab.

7. The method according to any of claim 7, wherein actarit and said another agent are provided in combination.

8. A pharmaceutical composition, for the treatment of inflammatory bowel disease, wherein said composition comprises actarit, or a salt thereof, with a pharmaceutical carrier.

9. The composition, according to claim 8, further comprising another agent used to treat inflammatory bowel disease.

10. The composition, according to claim 9, wherein said another agent is selected from the group consisting of steroids, salicylate drugs, immunosuppressants, antibiotics and Crohn's disease-neutralising antibodies.

11. The composition, according to claim 10, wherein said another agent is sulphasalasine, mesalamine, hydrocortisone, prednisolone, budesonide, azathioprine, 6-mercaptopurine, methotrexate, cyclosporine or infliximab.
